(19) United States
(12) Patent Application Publication
(10) Pub. No.: US 2004/0122464 A1
(43) Pub. Date: Jun. 24, 2004

(54) Balloon catheter having a microporous distal tip

(76) Inventors: Edwin Wang, Tustin, CA (US); Timothy Owens, Dublin, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/322,402

(22) Filed: Dec. 18, 2002

(51) Int. Cl. A61M 29/00

(52) U.S. Cl. 606/194

(57) ABSTRACT

A catheter having an elongated shaft having a proximal end, a distal end, at least one lumen therein, and a distal tip member, the distal tip member comprising a porous polymeric material. In one embodiment, the porous polymeric material is selected from the group consisting of expanded polytetrafluoroethylene, ultra high molecular weight polyolefin, porous polyurethane, porous nylon, porous polyethylene, porous polypropylene, and porous polyether block amide.

### BACKGROUND OF THE INVENTION

This invention generally relates to catheters, and particularly intravascular catheters for use in percutaneous transluminal coronary angioplasty (PTCA) or for the delivery of stents.

In percutaneous transluminal coronary angioplasty (PTCA) procedures a guiding catheter is advanced in the patient's vasculature until the distal tip of the guiding catheter is seated in the ostium of a desired coronary artery. A guidewire is first advanced out of the distal end of the guiding catheter into the patient's coronary artery until the distal end of the guidewire crosses a lesion to be dilated. A dilatation catheter, having an inflatable balloon on the distal portion thereof, is advanced into the patient's coronary anatomy over the previously introduced guidewire until the balloon of the dilatation catheter is properly positioned across the lesion. Once properly positioned, the dilatation balloon is inflated with inflation fluid one or more times to a predetermined size at relatively high pressures so that the stenosis is compressed against the arterial wall and the wall expanded to open up the vascular passageway. Generally, the inflated diameter of the balloon is approximately the same diameter as the native diameter of the body lumen being dilated so as to complete the dilatation but not overexpand the artery wall. After the balloon is finally deflated, blood flow resumes through the dilated artery and the dilatation catheter and the guidewire can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate of angioplasty alone and to strengthen the dilated area, physicians now normally implant an intravascular prosthesis, generally called a stent, inside the artery at the site of the lesion. Stents may also be used to repair vessels having an intimal flap or dissection or to generally strengthen a weakened section of a vessel or to maintain its patency. Stents are usually delivered to a desired location within a coronary artery in a contracted condition on a balloon of a catheter which is similar in many respects to a balloon angioplasty catheter, and expanded within the patient's artery to a larger diameter by expansion of the balloon. The balloon is deflated to remove the catheter and the stent left in place within the artery at the site of the dilated lesion. See for example, U.S. Pat. No. 5,507,768 (Lau et al.) and U.S. Pat. No. 5,458,615 (Klemm et al.), which are incorporated herein by reference.

An essential step in effectively performing a PTCA procedure is properly positioning the balloon catheter at a desired location within the coronary artery. To properly position the balloon at the stenosed region, the catheter shaft must be able to transmit force along the length of the catheter shaft to allow it to be pushed through the vasculature. However, the catheter shaft must also have sufficient flexibility to allow it to track over a guidewire through the often tortuous vasculature. Additionally, the catheter also must have good crossability (i.e., the ability of the catheter distal end to cross stenosed portions of the vascular anatomy).

Prior art intravascular catheters have commonly included a soft distal tip to prevent or minimize injury to the vessel during advancement of the catheter therein. One difficulty has been forming a connection between the soft tip and the catheter which is sufficiently strong to prevent disengagement of the soft tip or kinking at the junction between the soft tip and catheter shaft. Additionally, it is necessary to balance the strength of the connection between the soft tip and the catheter shaft with the need to minimize the stiffness of the distal end of the catheter. Minimizing the stiffness of the distal end of the catheter results in improved maneuverability of the catheter.

Accordingly, it would be a significant advance to provide a catheter with a soft tip having improved performance. This invention satisfies these and other needs.

### SUMMARY OF THE INVENTION

The invention is directed to a catheter which includes an elongated shaft having a proximal end, a distal end, at least one lumen therein, and a distal tip member, the distal tip member comprising a porous polymeric material.

In a presently preferred embodiment, the catheter is a balloon catheter having a balloon on a distal shaft section, with an interior in fluid communication with the at least one lumen of the catheter shaft. A balloon catheter of the invention generally comprises an elongated shaft having a proximal shaft section, a distal shaft section, an inflation lumen extending within the proximal and distal shaft sections, and a guidewire receiving lumen extending at least within the distal shaft section, and an inflatable balloon on the distal shaft section with an interior in fluid communication with the inflation lumen. The porous polymeric distal tip member typically defines a distal section of the guidewire lumen. In a presently preferred embodiment, the shaft comprises an outer tubular member defining the inflation lumen, and an inner tubular member defining a proximal section of the guidewire lumen in fluid communication with the distal section of the guidewire lumen defined by the distal tip member. The balloon catheter can have a variety of conventional configurations including an over-the-wire catheter in which the guidewire lumen extends from the proximal end of the shaft to a guidewire distal port at or near the distal end of the shaft, or a rapid exchange type catheter in which the guidewire lumen extends from the guidewire distal port to a proximal port spaced distally from the shaft proximal end. The balloon catheter of the invention may comprise a variety of suitable balloon catheters, including coronary and peripheral dilatation catheters, stent delivery catheters, drug delivery catheters, and the like.

In a presently preferred embodiment, the porous polymeric material of the distal tip member is expanded polytetrafluoroethylene (ePTFE). However, a variety of suitable porous materials may be used including porous fluoropolymers in general, an ultra high molecular weight polyolefin such as ultra high molecular weight polyethylene, porous polyurethane, porous nylon, porous polyethylene, and porous polypropylene, and porous polyether block amide. In one embodiment, the porous polymeric material has a node and fibril microstructure. For example, ePTFE and ultra high molecular weight polyethylene (also referred to as “expanded ultra high molecular weight polyethylene”) typically are microporous with a node and fibril microstructure. The porous polymeric material provides a distal tip with an improved, high longitudinal tensile strength of about 3,000 psi to about 8,000 psi. In a presently preferred embodiment, the porous polymeric material is lubricious, to facilitate advancement of the catheter within the body lumen and/or advancement of a guidewire within the distal tip lumen. For example, ePTFE and ultra high molecular weight polyethylene are relatively highly lubricious. In one embodiment, the porous polymeric material of the distal tip has a lower coefficient of friction than the polymeric material forming at least a surface (i.e., inner and/or outer surface) of the shaft inner tubular member located proximal to the distal tip member.

The microporous structure of the polymeric material forming the distal tip is such that the material is typically gas permeable. However, fluids such as body fluids in the patient's body lumen and contrast fluid in the catheter lumen preferably do not permeate through the porous material absent significant pressurization forcing the fluid therethrough. In one embodiment, the microporous structure has a pore size distribution of about 1.5 to about 30 micrometers, preferably about 3 to about 15 micrometers. Thus, the pores of the porous polymeric material are typically substantially smaller than ports such as perfusion or device ports which may be formed in a sidewall of distal shaft sections of balloon catheters, and in one embodiment, the porous distal tip member has a continuous cylindrical wall around the circumference of the distal tip member (i.e., no side-wall ports) extending from the proximal end to a distal end of the distal tip member, unlike prior art perfusion catheters having side-wall ports in a distal shaft section.

The distal tip member is typically bonded to a distal skirt section of the balloon and/or the distal end of the shaft section defining the guidewire lumen proximal to the distal tip member (e.g., the inner tubular member). In one embodiment, the distal tip member has a surface which is etched or otherwise treated to improve the ability of the porous polymeric material to bond to an adjacent catheter component. For example, in the embodiment in which the distal tip member is formed of a porous fluoropolymer including ePTFE, the porous fluoropolymeric material is preferably etched or otherwise modified to improve bondability of the porous fluoropolymer. The surface treatments include chemical etching, plasma treatment, and deposition of a plasma polymerized species. In one embodiment, a distal portion of the outer surface of the distal tip member bonded to the balloon has an etched surface for improved bondability, and a proximal portion which is not etched so that the lubricity of the proximal portion is not reduced.

The porous polymeric material of the distal tip member provides an improved atraumatic distal tip with a low profile and high longitudinal tensile strength. Additionally, in one embodiment, the distal tip is highly lubricious without requiring a lubricious coating applied to the tip. Moreover, one embodiment having an ePTFE distal tip bonded to an ePTFE layer of a balloon, the ePTFE distal tip has a strong bond to the ePTFE layer of the balloon. These and other advantages of the invention will become more apparent from the following detailed description and exemplary drawings.

### BRIEF DESCRIPTION OF THE DRAWINGS

FIG. 1 is an elevational view, partially in section, of a balloon catheter which embodies features of the invention.

FIG. 2 is a transverse cross sectional view of the catheter shown in FIG. 1, taken along line 2-2.

FIG. 3 is a transverse cross sectional view of the catheter shown in FIG. 1, taken along line 3-3.

FIG. 4 is a transverse cross sectional view of the catheter shown in FIG. 1, taken along line 4-4.

### DETAILED DESCRIPTION OF THE INVENTION

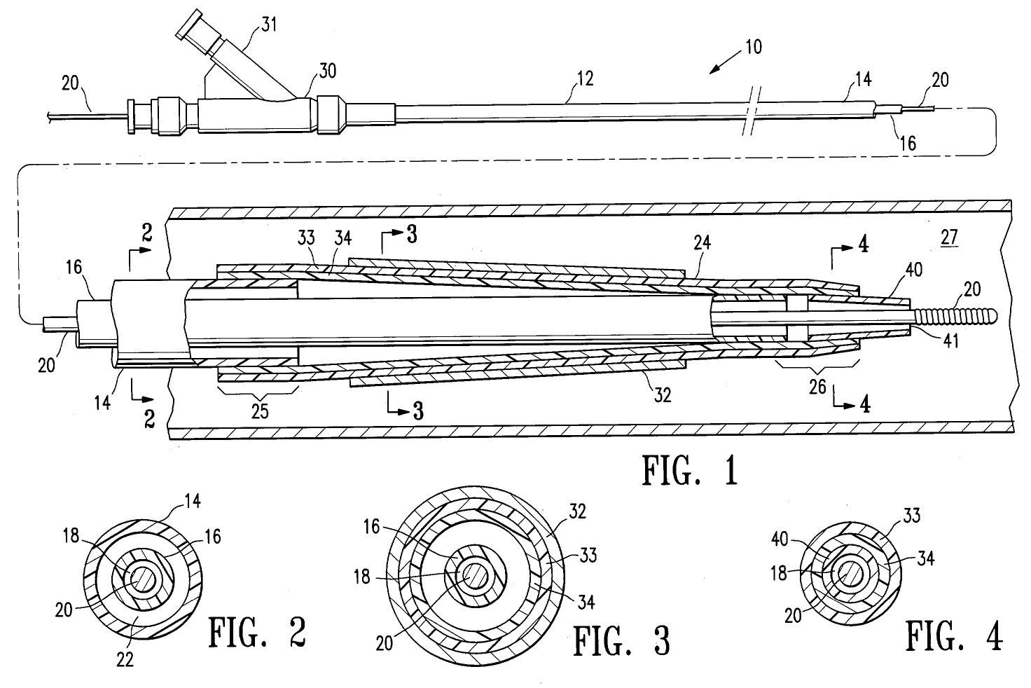

FIG. 1 illustrates a balloon catheter 10 which embodies features of the invention. Catheter 10 generally comprises an elongated catheter shaft 12 having an outer tubular member 14 and an inner tubular member 16. Inner tubular member 16 defines a guidewire lumen 18 configured to slidingly receive a guidewire 20, and the coaxial relationship between outer tubular member 14 and inner tubular member 16 defines annular inflation lumen 22, as best shown in FIGS. 2 and 3 illustrating a transverse cross section views of the catheter shown in FIG. 1, taken along lines 2-2 and 3-3, respectively. An inflatable balloon 24 disposed on a distal section of catheter shaft 12 has a proximal skirt section 25 sealingly secured to the distal end of outer tubular member 14 and a distal skirt section 26 sealingly secured to the distal end of inner tubular member 16, so that its interior is in fluid communication with inflation lumen 22. An adapter 30 at the proximal end of catheter shaft 12 is configured to provide access to guidewire lumen 18, and to direct inflation fluid through arm 31 into inflation lumen 22. FIG. 1 illustrates the balloon 24 in a low profile tubular configuration prior to complete inflation, with an expandable stent 32, mounted on the balloon 24 for implanting within a patient's body lumen 27. The distal end of catheter 10 may be advanced to a desired region of the patient's body lumen 27 in a conventional manner, and balloon 24 fully inflated to expand stent 32, and the balloon deflated, leaving stent 32 implanted in the body lumen 27.

A distal tip member 40 forms the distal end of the shaft 12 and defines a distal section of guidewire lumen 18 in fluid communication with the proximal section of the guidewire lumen 18 defined by the inner tubular member 16. A guidewire distal port 41 at the distal end of the catheter is defined by the distal tip member 40. In the embodiment of FIG. 1, the distal tip member 40 has a circular transverse shape, as best shown in FIG. 4 illustrating a transverse cross section view of the distal end of the catheter shown in FIG. 1, taken along line 4-4. However, the distal tip member 40 may have a variety of suitable shapes as are conventionally known such as an oblong transverse cross section.

The distal tip member 40 is formed of a porous polymeric material, which in one presently preferred embodiment is commercially available expanded polytetrafluoroethylene having a porosity of about 50 to about 90 percent, although a variety of commercially available polymeric materials may be used. The ePTFE porous polymeric material has a low coefficient of friction, providing the tip with a lubricious surface. In another presently preferred embodiment, the porous polymeric material of the distal tip member is commercially available ultra high molecular weight polyethylene having a porosity of about 30 to about 50 percent. The porosity of the polymeric material forming the distal tip member 40 is the result of conventionally known methods of producing the porosity typically involving expanding a sheet of the polymeric material.

The polymeric material forming distal tip member 40 is preferably softer and more flexible than the polymeric inner tubular member 16, so that the distal tip member 40 is more flexible than the inner tubular member 16. The inner tubular member 16 typically has at least one layer formed of a polymeric material with a higher Shore durometer hardness than the polymeric material of the distal tip member 40, such as for example a polyamide such as polyether block amide (PEBAX), available from Autochem, and nylons.

In the embodiment illustrated in FIG. 1, the distal tip member 40 is tapered, with an inner and an outer surface tapering distally to a smaller inner and outer diameter, respectively. In a presently preferred embodiment, the distal tip member 40 tapers from an outer diameter of about 0.028 to about 0.017 inches (0.7 to 0.45 mm), and an inner diameter of about 0.018 to about 0.016 inches (0.46 to 0.41 mm). In the embodiment in which the tip member 40 polymeric material is ePTFE or ultra high molecular weight polyethylene, the tapering tip member 40 is preferably formed by wrapping a sheet of the polymeric material on a tapered mandrel and heating to fuse the wrapped sheet together, or by ram extrusion.

The distal tip member 40 preferably has a wall thickness less than or equal to the wall thickness of the section of the inner tubular member 16 proximally adjacent thereto. The wall thickness of the distal tip member 40 is typically about 0.001 to about 0.003 inches (0.025 to 0.075 mm). The distal tip member 40 preferably has a length which is significantly shorter than the length of the inner tubular member, and specifically about 0.1 to about 5% of the length of the inner tubular member 15. The length of the tip member 40 is typically about 1 to about 6 mm, and more preferably about 2 to about 4 mm.

In the embodiment of FIG. 1, the proximal end of the tip member 40 is spaced distally from the distal end of the inner tubular member 16, with a gap therebetween. However, a variety of suitable configurations can be used including securing the distal tip member 40 to the distal end of the inner tubular member 16 with a butt or lap joint. The balloon distal skirt section 26 sealingly surrounds and is bonded to the distal end of the inner tubular member 16 and the proximal end of the distal tip member 40 in the embodiment of FIG. 1. The distal end of the distal tip member 40 is located distal to the distal end of the balloon distal skirt section 26.

The distal tip member 40 and inner tubular member 16 are preferably bonded to the balloon distal skirt section 26 by fusion bonding, although adhesive may be used in addition to or as an alternative to fusion bonding. In a presently preferred embodiment in which the distal tip member 40 is formed of ePTFE, at least a portion of the outer surface of the ePTFE distal tip member 40 is etched by a chemical etch or modified by a plasma treatment process before bonding. Preferably, only the proximal portion of the distal tip member 40 bonded to the balloon distal skirt section 26 is etched, so that the lubricity of the exposed distal outer surface of the distal tip member 40 is not reduced by etching. The length of the etched portion is typically about 10% to about 50% of the length of the distal tip member 40.

In the embodiment illustrated in FIG. 1, balloon 24 has a first layer 33 and a second layer 34. In a presently preferred embodiment, the balloon 24 first layer 33 comprises a porous polymeric material, and preferably a microporous polymeric material having a node and fibril microstructure, such as ePTFE. In the embodiment illustrated in FIG. 1, first layer 33 is formed of ePTFE, and the second layer 34 is formed of a polymeric material preferably different from the polymeric material of the first layer 33. Although discussed below in terms of one embodiment in which the first layer 33 is formed of ePTFE, it should be understood that the first layer may comprise other materials, including ultrahigh molecular weight polyethylene. The second layer 34 is preferably formed of an elastomeric material, such as polyurethane elastomers, silicone rubbers, dienes, styrene-butadiene-styrene block copolymers, polyamide block copolymers, and the like. In a preferred embodiment, layer 34 is an inner layer relative to layer 33, although in other embodiments it may be an outer layer. Layer 34 limits or prevents leakage of inflation fluid through the microporous ePTFE to allow for inflation of the balloon 24, and expands elastically to facilitate deflation of the balloon 24 to a low profile deflated configuration. The layer 34 may consist of a separate layer which neither fills the pores nor disturbs the node and fibril structure of the ePTFE layer 33, or it may at least partially fill the pores of the ePTFE layer.

Although illustrated in the embodiment of FIG. 1 having the ePTFE outer layer 33 of the balloon 24 separated from the distal tip member 40 by the inner layer 34 of the balloon 24, in an alternative embodiment (not shown), the ePTFE outer layer 33 extends beyond the distal end of the inner layer 34 and onto at least a portion of the outer surface of the distal tip member 40 and is bonded thereto. In the embodiment in which the catheter balloon has a porous polymeric layer (e.g., layer 33), the porous polymeric layer of the balloon is preferably formed of the same porous polymeric material as the distal tip member 40, for improved bondability thereto.

To the extent not previously discussed herein, the various catheter components may be formed and joined by conventional materials and methods. For example, inner tubular member 16 can be formed by conventional techniques, such as by extruding and necking materials found useful in intravascular catheters such as polyethylene, polyvinyl chloride, polyesters, polyamides, polyimides, polyurethanes, and composite materials, and is preferably a multilayered tubular member. Additionally, although not illustrated, coiled or braided reinforcements may be included in the shaft at various locations, as is conventionally known.

The length of the dilatation catheter 10 is generally about 108 to about 200 centimeters, preferably about 137 to about 145 centimeters, and typically about 143 centimeters for PTCA. The outer tubular member 14 distal section has an outer diameter (OD) of about 0.028 to about 0.036 inch (0.70-0.91 mm), and an inner diameter (ID) of about 0.024 to about 0.035 inch (0.60-0.89 mm), and the outer tubular member 14 proximal section has an OD of about 0.036 to about 0.042 inch (0.9-1 mm), and an inner diameter (ID) of about 0.034 to about 0.036 inch (0.86-0.9 mm). The inner tubular member 16 has an OD of about 0.017 to about 0.026 inch (0.43-0.66 mm), and an ID of about 0.015 to about 0.02 inch (0.38-0.5 mm) depending on the diameter of the guidewire to be used with the catheter. The balloon 24 has a length of about 14 mm to about 46 mm, and an inflated working diameter of about 8 mm to about 40 mm.

While the present invention has been described herein in terms of certain preferred embodiments, those skilled in the art will recognize that modifications and improvements may be made without departing from the scope of the invention. For example, although the catheter 10 illustrated in the Figures is an over-the-wire balloon catheter, the catheter of the invention may be a variety of suitable catheters, including other balloon catheter configurations, or guiding catheters, and the like. Additionally, although discussed primarily in terms of the embodiment in which the guidewire is slidably disposed in the guidewire lumen, a variety of conventional balloon catheter configurations can be used, including fixed-wire catheters in which the guidewire is fixable within a distal portion of the guidewire lumen. While individual features of one embodiment of the invention may be discussed or shown in the drawings of the one embodiment and not in other embodiments, it should be apparent that individual features of one embodiment may be combined with one or more features of another embodiment or features from a plurality of embodiments

What is claimed is:

1. A catheter comprising an elongated shaft having a proximal end, a distal end, at least one lumen, and a distal tip member, the distal tip member comprising a porous polymeric material.

2. The catheter of claim 1 wherein the porous polymeric material is selected from the group consisting of expanded polytetrafluoroethylene, ultra high molecular weight polyolefin, porous polyurethane, porous nylon, porous polyethylene, porous polypropylene, and porous polyether block amide.

3. A balloon catheter, comprising:
a) an elongated shaft having a proximal end, a distal end, an inflation lumen, a guidewire lumen, and a distal tip member defining a distal section of the guidewire lumen, the distal tip member comprising a porous polymeric material and having a proximal end located distal to the proximal end of the shaft; and
b) a balloon on a distal shaft section, having an interior in fluid communication with the inflation lumen of the shaft.

4. The catheter of claim 3 wherein the distal tip member defines a guidewire distal port at the distal end of the shaft.

5. The catheter of claim 3 wherein the shaft comprises an outer tubular member defining the inflation lumen, and an inner tubular member disposed in at least a distal section of the inflation lumen and defining a section of the guidewire lumen in fluid communication with the guidewire lumen distal section defined by the distal tip member.

6. The catheter of claim 3 wherein the distal tip member defines the distal end of the shaft.

7. The catheter of claim 3 wherein the distal tip member has a tubular body defining an inner surface and an outer surface of the distal tip member and formed of the porous polymeric material.

8. The catheter of claim 3 wherein the distal tip member has a continuous cylindrical wall around the circumference of the distal tip member, extending from the proximal end to a distal end of the distal tip member.

9. The catheter of claim 3 wherein the porous polymeric material has a microporous structure with a pore size distribution of about 1 to about 30 micrometers.

10. The catheter of claim 3 wherein the porous polymeric material has a node and fibril microstructure.

11. The catheter of claim 3 wherein the porous polymeric material is selected from the group consisting of expanded polytetrafluoroethylene, ultra high molecular weight polyolefin, porous polyurethane, porous nylon, porous polyethylene, porous polypropylene, and porous polyether block amide.

12. The catheter of claim 3 wherein the porous polymeric material is lubricious.

13. The catheter of claim 3 wherein the porous polymeric material of the distal tip member has an etched surface bonded to an adjacent catheter component.

14. The catheter of claim 13 wherein the adjacent catheter component is selected from the group consisting of the catheter balloon and a section of the shaft extending at least in part proximally of the distal tip member.

15. The catheter of claim 3 wherein the distal tip member is formed of expanded polytetrafluoroethylene.

16. The catheter of claim 15 wherein the balloon has at least one layer formed of expanded polytetrafluoroethylene and bonded to the distal tip member.

17. A balloon catheter, comprising:
a) an elongated shaft having a proximal end, a distal end, an inflation lumen, a guidewire receiving lumen, and a distal tip member comprising a porous polymeric material selected from the group consisting of expanded polytetrafluoroethylene and ultrahigh molecular weight polyethylene, the distal tip member defining a distal section of the guidewire lumen; and
b) a balloon on a distal shaft section, having an interior in fluid communication with the inflation lumen of the shaft.

18. The catheter of claim 17 wherein the shaft comprises an outer tubular member defining the inflation lumen, and an inner tubular member in at least a distal section of the outer tubular member defining a section of the guidewire lumen in fluid communication with a distal section of the guidewire lumen defined by the distal tip member.

19. The catheter of claim 18 wherein the distal tip member is bonded to a distal end of the inner tubular member and defines the distal end of the shaft.
